(19) United States
(12) Patent Application Publication
(10) Pub. No.: US 2026/0240893 A1
(43) Pub. Date: Aug. 20, 2026

(54) STABLE AQUEOUS READY-TO-USE COMPOSITION OF HYDROXOCOBALAMIN

(71) Applicants: Extrovis AG, Baar (CH); Extrovis Private Limited, Hyderabad (IN)

(72) Inventors: Janos Vaczi, Kuessnacht am Rigi (CH); Hanumantha Rao Kamma, Baar (CH); Rajasekhara Reddy Singampalli, Hyderabad (IN)

(73) Assignees: Extrovis AG, Baar (CH); Extrovis Private Limited, Hyderabad (IN)

(21) Appl. No.: 19/164,215

(22) PCT Filed: Mar. 15, 2024
(86) PCT No.: PCT/IN2024/050271

§ 371 Date: Sep. 11, 2025

(30) Foreign Application Priority Data
Mar. 18, 2023 (IN) 202321018456

(51) Int. Cl. A61K 31/714; A61K 9/08; A61K 47/02; A61K 47/12

(52) U.S. Cl. CPC A61K 31/714; A61K 9/08; A61K 47/02; A61K 47/12

(57) ABSTRACT

The present invention provides a stable aqueous ready-to-use composition of hydroxocobalamin for parenteral use comprising hydroxocobalamin as the sole active ingredient. The ready-to-use composition does not require reconstitution and is stable over its shelf life.

### FIELD OF THE INVENTION

The present invention relates to a stable aqueous ready-to-use composition of hydroxocobalamin for parenteral use comprising hydroxocobalamin as the sole active ingredient. The ready-to-use composition saves time in treating the patient, as time required for reconstitution is saved, and a faster remedy is provided to the patient.

### BACKGROUND OF THE INVENTION

Hydroxocobalamin is a synthetic, injectable form of Vitamin B12 (I). Hydroxocobalamin is actually a precursor of two cofactors or vitamins (Vitamin B12 and methylcobalamin) which are involved in various biological systems in a human being. The IUPAC name for hydroxocobalamin is Coα-[α-(5,6-dimethylbenzimidazolyl)]-Coβ-hydroxocobamide. It has been used therapeutically to treat vitamin B12 deficiency. It is also used in treating cyanide poisoning, Leber's optic atrophy, pernicious anaemia and toxic amblyopia.

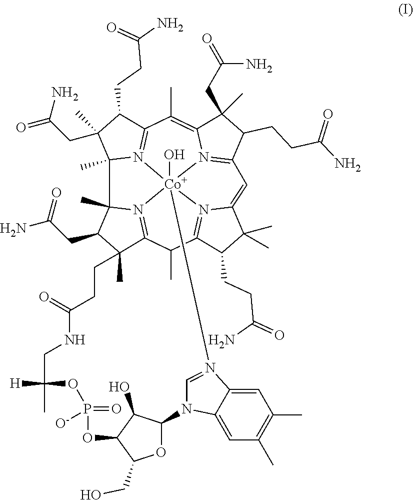

The product is commercially available in the United States as CYANOKIT (hydroxocobalamin for injection) for intravenous infusion, which is a cyanide antidote package that contains one colourless 250 mL glass vial containing 5 g dark red lyophilized hydroxocobalamin, pH adjusted with hydrochloric acid, one transfer spike, one intravenous administration set, one quick use reference guide and one package insert. The 5 g vial of hydroxocobalamin for injection is to be reconstituted with 200 mL of 0.9% sodium chloride solution, to give a dark red injectable solution (25 mg/mL). If 0.9% sodium chloride is not readily available, 200 mL of either Lactated Ringers injection or 5% Dextrose injection (D5W) may be used as the diluent. Diluent is not included in the CYANOKIT. The pH of the reconstituted product ranges from 3.5 to 6.0.

As the diluent is not provided with the kit, there is a need to carry 200 ml of reconstitution solution along with the vial. The volume of the solution makes the kit bulkier making it difficult to carry and store. Further, reconstitution process could lead to errors, thereby leading to under or overdosing (dosing errors). It must also be noted that a lyophilized product that requires reconstitution, followed by withdrawal of desired dose and dilution with large volume parenteral, leads to creation of significant amount of waste, such as syringes, vials, gloves, needles, masks and equipment, and also needs longer time in preparing the solution and making it available for administration to the patient. Hydroxocobalamin solution, as described above, is usually used for people working on war-front or in the defense services. In a life-threatening situation, where a quick treatment is required, if the solvent is not available for reconstitution, and/or if it takes longer to reconstitute and prepare the solution, and/or if there are resulting errors in dosing, the patient may succumb to death due to poisoning.

In light of the above, there is a need for ready-to-use composition of hydroxocobalamin. This would avoid the step of reconstitution and handling. In order to make such compositions, a suitable solvent system is essential, along with a packaging system that is robust and maintains the quality and purity of the product over a long period of time. The solution also would need to be sterile for use as a parenteral, and would need to be so preserved until it is administered to the patient. Further, hydroxocobalamin must not degrade in the solution and must remain stable throughout its shelf life.

US20220257635 relates to an injectable nutritional supplement for livestock and in particular an aqueous injectable nutritional supplement for livestock containing vitamin B12 and one or more trace elements like zinc, copper, selenium. The document teaches the use of organic solvents such as propylene glycol to provide stable formulations. Further, the document demonstrates that a lower pH does not enhance the stability of Vitamin B12 solution.

U.S. Pat. No. 9,585,908 (granted on Mar. 7, 2017) relates to a collyrium composition comprising hydroxocobalamin, hydroxo (aquo) cobalamin or a mixture of hydroxocobalamin and hydroxo (aquo) cobalamin, the mixture sometimes referred to therein in a shorthand manner as hydroxocobalamin. The patent describes use of the composition for substantially deactivating reactive pollutant combustion gases adsorbed on eye surfaces, periocular tissues and gas permeable contact lenses. This patent does not disclose parenteral formulations of hydroxocobalamin.

U.S. Pat. No. 3,291,691 discloses the synergy of hydroxocobalamin and sodium thiosulphate parenteral formulations for the treatment of cyanide poisoning.

U.S. Pat. No. 3,161,568 relates to stable aqueous solutions of hydroxocobalamin for parenteral administration. Further, the patent discloses that the use of oxygen free atmosphere enhances the stability of the solution. However, it is to be noted that the aqueous formulations of hydroxocobalamin disclosed herein contain a preservative which is a mixture of methylpara hydroxybenzoate and propylpara-hydroxybenzoate in a weight ratio of 5:1 to 10:1.

US2008039422 discloses compositions that include one or more vitamin B12 compounds and one or more excipients that enhance solubility of the vitamin B12 compounds. However, the document teaches the use of co-solvents propylene glycol and ethanol in the formulation to increase the solubility and stability of Vitamin B12 compounds, and a pH of 8.9 for maximum solubility. Further, prior to administration, the formulation requires heating to solubilize the compound in the excipients or solvents.

WO2022063963 (Published on Mar. 31, 2022) relates to an invention about a terminally heat-sterilized pre-prepared and pre-packaged product for parenteral nutrition which can be used for preventing or correcting vitamin B12 deficiency in a patient comprising a formulation for parenteral administration provided in a flexible container comprising vitamin B12. The publication does not, however, provide details on the long-term stability of the described formulations. A comparison of a formulation of this application with that of the present invention is provided in example 3 below.

### OBJECT OF THE INVENTION

It is an object of the invention to provide a stable ready-to-use composition of hydroxocobalamin that is sterile, aqueous and that does not require reconstitution.

Another object of the invention is to provide a ready-to-use composition of hydroxocobalamin that is essentially free of preservatives, co-solvents, polymers, chelating agents and reconstitution or dilution agents.

A further object of the invention is to provide a ready-to-use composition of hydroxocobalamin that is stable over a long period of time at different temperatures.

Yet another object of the invention is to provide a ready-to-use composition of hydroxocobalamin that can be administered directly to a patient thereby saving time otherwise required for reconstitution and/or dilution.

These and other objects and advantages of the invention will be apparent from the ensuing description.

### SUMMARY OF THE INVENTION

The present invention relates to a stable ready-to-use composition of hydroxocobalamin that is sterile, aqueous and that does not require reconstitution. The hydroxocobalamin composition of the present invention is useful in treating cyanide poisoning, and can be administered directly to the patient without spending any time on reconstitution and/or dilution.

The present invention relates to a stable sterile aqueous ready-to-use pharmaceutical composition for parenteral use comprising hydroxocobalamin or its pharmaceutically acceptable salt as the sole active ingredient, a tonicity adjusting agent in an amount sufficient to provide osmolality of about 240 mOsm/kg to about 360 mOsm/kg, and a pH modifying agent. The composition, upon storage at room temperature, has not more than about 0.5% of impurity B6-Hydroxymethyl derivative, not more than about 0.4% of impurity B5-Hydroxymethyl derivative, not more than about 1% of C8-epimer impurity, not more than about 1% of cyanocobalamin, not more than about 0.2% of any unspecified impurity, and not more than about 6% of total impurities.

In a preferred embodiment, the ready-to-use composition of the present invention comprises hydroxocobalamin or its pharmaceutically acceptable salt, a tonicity adjusting agent, and a pH modifying agent wherein the composition is essentially free of preservatives, co-solvents, polymers, chelating agents and reconstitution or dilution agents. In a preferred embodiment, the ready-to-use composition of the present invention containing hydroxocobalamin or its pharmaceutically acceptable salt comprises a tonicity adjusting agent selected from the group comprising sodium chloride, potassium chloride, mannitol, lactose, dextrose, glycerol and mixtures thereof.

In another preferred embodiment, the ready-to-use composition of the present invention containing hydroxocobalamin or its pharmaceutically acceptable salt comprises a pH-modifying agent. The pH-modifying agent is selected from the group comprising acetic acid, tartaric acid, lactic acid, hydrochloric acid, citric acid, succinic acid, fumaric acid, maleic acid, malic acid, gluconic acid, their pharmaceutically acceptable salts and mixtures thereof.

In another preferred embodiment, the ready-to-use composition of the present invention containing hydroxocobalamin or its pharmaceutically acceptable salt has a pH in the range of about 3.5 to about 6; preferably about 4 to about 5.

In a preferred embodiment, the ready-to-use composition of the present invention containing hydroxocobalamin or its pharmaceutically acceptable salt is stable over a period of 18 months when stored at about 2° C. to about 8° C., or at controlled room temperature.

In another preferred embodiment, the composition is stored at 2-8° C. or at controlled room temperature in a container comprising a primary packaging made of polypropylene homopolymer, wherein the primary packaging of the container is a polypropylene bag with a twist off port, or a polypropylene vial.

In a preferred embodiment, the ready-to-use composition of the present invention containing hydroxocobalamin or its pharmaceutically acceptable salt is stored in a container comprising a secondary packaging which is a tri-laminated aluminium pouch of a size suitable of accommodating the primary packaging.

### DETAILED DESCRIPTION OF THE INVENTION

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

Unless the context indicates otherwise, it is specifically intended that the various features of the invention described herein can be used in any combination.

Moreover, the present invention also contemplates that in some embodiments of the invention, any feature or combination of features set forth herein can be excluded or omitted.

All publications, patent applications, patents, and other references mentioned herein are incorporated by reference herein in their entirety for all purposes.

The term “comprising”, which is synonymous with “including”, “containing”, or “characterized by” herein defined as being inclusive or open-ended and does not exclude additional, unrecited elements or method steps, unless the context clearly requires otherwise.

As used herein, “a,” “an,” or “the” can mean one or more than one. For example, “a” solvent can mean a single solvent or a multiplicity of solvents.

Also as used herein, “and/or” refers to and encompasses any and all possible combinations of one or more of the associated listed items, as well as the lack of combinations when interpreted in the alternative (“or”).

Furthermore, the term “about,” as used herein when referring to a measurable value such as an amount of a compound, related compound, impurity or agent of this invention, dose, time, temperature, and the like, is meant to encompass variations of ±10% of the specified amount.

The term “essentially free of” specifies that the content of a particular excipient is minimal and falls below certain thresholds. Specifically, it indicates that the amount of the excipient is less than 5 wt % (weight percent), preferably less than 4 wt %, even more preferably less than 3 wt %, and most preferably less than 2 wt %. In some instances, it may even be as low as 1 wt %, 0.1 wt %, 0.05 wt %, or 0.01 wt %.

The present invention provides a sterile ready-to-use composition of hydroxocobalamin or its pharmaceutically acceptable salt that is stable, and which provides the advantages of reduced dosing errors and ease of administration.

As used herein, and unless otherwise specified, the term “stable” refers to physical and chemical stability of the composition over the shelf-life of the composition. The United States Pharmacopoeia (USP) defines stability as “the ability of a product to retain its characteristics that it possessed during its manufacturing (physical, chemical, microbiological, therapeutic properties) within specified limits throughout its period of storage and use”. According to the ICH guidelines, pharmaceutical stability testing is defined as “systematic experiments conducted on pharmaceutical products to understand and provide evidence how the quality of a drug product varies under the influence of variety of environmental factors such as temperature, humidity and light, and to set re-test period for the drug, or a shelf life for the drug product and recommend good storage conditions”. In view of these, degradation products in a drug product are required to be evaluated and reported to the regulatory agencies. Further, USFDA requires parenteral formulations to be stable for a minimum period of 12 months (i.e. 48 weeks) at recommended storage condition, for approving the same for commercial use. For products to be commercially marketed in the US, there is an expectation that the products will comply with the standard throughout the shelf life.

Thus, the composition must have acceptable level of impurity. The term “acceptable level of impurity” refers to the impurities in drug products not exceeding the permitted daily exposure (PDE), and/or impurities within limits defined by ICH for pharmaceutical products.

The term “controlled room temperature” is within the range of 20° C. to 25° C. as per US Pharmacopeia guidelines (USP <659>).

The term “shelf life” refers to the amount of time the ready-to-use composition of the present invention may be stored without loss of potency and/or performance profile. In some embodiments of the present invention, shelf life refers to the amount of time the ready-to-use composition may be stored with loss of not more than about 6%, preferably not more than about 4%, preferably note more than about 3%, preferably not more than about 2% or still preferably not more than about 1% of the potency and/or performance, when stored at room temperature of 25° C. and 40% relative humidity. The stable composition provided herein is designed to have shelf life of at least about 12 months, preferably about 15 months, more preferably about 18 months, even more preferably about 24 months.

The term “sterile” refers to the pharmaceutical composition which is free from contamination caused by harmful bacteria, viruses, or other microorganisms wherein the pharmaceutical composition is surgically sterile or sterilized.

The present invention therefore provides a stable sterile aqueous composition of hydroxocobalamin or its pharmaceutically acceptable salt comprising a therapeutically effective amount of hydroxocobalamin, a tonicity adjusting agent and a pH modifying agent, wherein the composition is stable over its shelf-life. The composition of the present invention is stable when stored at lower temperatures (2-8° C.), as well as at controlled room temperature over its shelf life.

The stable sterile aqueous composition of the present invention contains hydroxocobalamin or its pharmaceutically acceptable salt in an amount ranging from about 0.1% w/v to about 5% w/v of the composition. Typically, the stable aqueous ready-to-use composition of the present invention contains 25 mg/ml (2.5%) of hydroxocobalamin. In preferred embodiments of the present invention, the stable aqueous ready-to-use composition of hydroxocobalamin has a volume of 200 ml. In still preferred embodiments, the present invention provides stable aqueous ready-to-use composition containing 25 mg/ml of hydroxocobalamin in a volume of 200 ml.

The tonicity adjusting agent present in the stable sterile aqueous ready-to-use composition of the present invention is used in an amount such that it is sufficient to provide an osmolality of about 240 mOsm/kg to about 360 mOsm/kg. The tonicity adjusting agent may be selected from the group comprising sodium chloride, potassium chloride, mannitol, lactose, dextrose, glycerol and mixtures thereof. In a preferred embodiment, sodium chloride is used as the tonicity adjusting agent in an amount sufficient to provide osmolality of about 240 mOsm/kg to about 360 mOsm/kg. In a still preferred embodiment, the stable aqueous composition of the present invention contains sodium chloride as the tonicity adjusting agent in an amount ranging from about 0.5% w/v to about 1.0% w/v of the composition.

The present invention provides a stable sterile aqueous ready-to-use composition of hydroxocobalamin comprising one or more pH adjusting agents selected from the group comprising acetic acid, tartaric acid, lactic acid, hydrochloric acid, citric acid, succinic acid, fumaric acid, maleic acid, malic acid, gluconic acid, their pharmaceutically acceptable salts and mixtures thereof. The stable aqueous ready-to-use composition of the present invention has a pH in the range of about 3.5 to about 6; preferably about 4 to about 5. The amount of the pH adjusting agent used is such that it is sufficient to provide pH in the range of about 3.5 to about 6.

The prior art provided teachings for using cosolvents (US20220257635) and/or polymers and chelating agents (example of WO2022063963), to obtain solutions of hydroxocobalamin. The present invention does not use any cosolvents, polymers or chelating agents in the composition, and yet is stable and sterile over its shelf life.

Typically, parenteral preparations contain preservatives to inhibit the growth of micro-organisms that may be introduced during processing. However, there are no preservatives available that possess sufficient efficacy, safety and non-irritancy to allow their inclusion in parenteral products. There is also mounting pressure from regulatory agencies and medical fraternity to develop preservative free parenteral products. The present invention provides stable aqueous ready-to-use compositions of hydroxocobalamin that are free of preservatives, and wherein the compositions are sterile over the entire shelf life. The compositions may be terminally sterilized, or may be prepared by aseptic manufacturing processes, as further described below.

Parenteral drug products must be sterile so as to ensure patient safety. Typically, a sterile ready-to-use composition of the present invention can be obtained by (1) terminally sterilizing the composition in its final container, using moist heat sterilization, dry heat sterilization or radiation sterilization, or (2) by manufacturing and filling the ready-to-use composition in an aseptic manner, ensuring that the infrastructure used and the processes employed do not contribute to introduction of microbial contamination at any time. A person of skill in the art is aware of these methods and how these are to be carried out.

In preferred embodiments, the aqueous ready-to-use composition of the present invention is manufactured using aseptic processing and sterile filtration. In one embodiment, the ready-to-use composition is filtered using polyvinylidene fluoride (PVDF) sterile filter having pore size of 0.22μ.

In another preferred embodiment, the ready-to-use composition of the present invention is terminally sterilized. In this method, the sterilization takes place after formulation, and once the product has been filled into the primary packaging. The terminal sterilization of pharmaceutical composition is preferably performed with saturated steam/super-heated water/counter pressure at 121° C. in an autoclave for at least 15 minutes.

As discussed above, the hydroxocobalamin ready-to-use composition of the present invention is stable over its shelf life. The specification for impurities in the ready-to-use composition is as seen in the Table 1 below—

TABLE 1
Impurity specification at shelf-life for ready-to
use compositions of the present invention

| | Specification |
|---|---|
| Impurity | (NMT* %) |
| B6-Hydroxymethyl derivative | 0.5 |
| B5-Hydroxymethyl derivative | 0.4 |
| C8-epimer of Hydroxocobalamin | 1.0 |
| Cyanocobalamin | 1.0 |
| Unspecified Impurity | 0.2 |
| Total Impurities | 6.0 |

*NMT = Not more than

The impurities listed in Table 1 above are described in Table 2 below.

TABLE 2

| Name of | | |
|---|---|---|
| Impurity | Chemical name of the impurity | Structure |
| B6-Hydroxy- methyl derivative | Cobalt(2+);[(2R,3S,4R,5S)-5-(5methyl, 6- Hydroxy methylbenzimidazol-1-yl)-4-hydroxy- 2-(hydroxymethyl)oxolan-3-yl][(2R)-1-[3- [(1R,2R,3R,4Z,7S,9Z,12S,13S,14Z,17S,18S,19R)- 2,13,18-tris(2-amino-2-oxoethyl)-7,12,17- tris(3-amino-3-oxopropyl)-3,5,8,8,13,15,18,19- octamethyl-2,7,12,17-tetrahydro-1H-corrin-21- id-3-yl]propanoylamino]propan-2-yl]phosphate; hydrate | 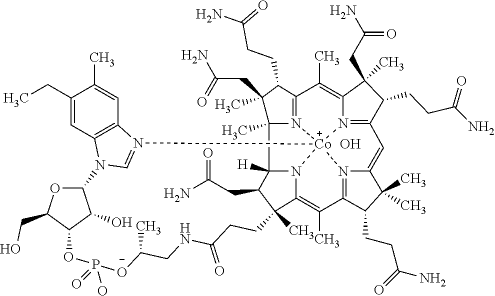 |
| B5-Hydroxy- methyl derivative | Cobalt(2+);[(2R,3S,4R,5S)-5-(5-Hydroxy methyl, 6-methylbenzimidazol-1-yl)-4-hydroxy- 2-(hydroxymethyl)oxolan-3-yl] [(2R)-1-[3- [(1R,2R,3R,4Z,7S,9Z,12S,13S,14Z,17S,18S,19R)- 2,13,18-tris(2-amino-2-oxoethyl)-7,12,17- tris(3-amino-3-oxopropyl)-3,5,8,8,13,15,18,19- octamethy1-2,7,12,17-tetrahydro-1H-corrin-21- id-3-yl]propanoylamino]propan-2-yl]phosphate; hydrate | 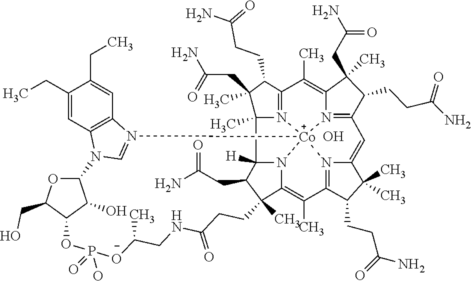 |
| C8-epimer of Hydroxo- cobalamin | Cobalt(3+);[(2R,3S,4R,5S)-5-(5,6- dimethylbenzimidazol-1-yl)-4-hydroxy-2- (hydroxymethyl)oxolan-3-yl][(2R)-1-[3- [(2R,3R,4Z,7S,9Z,12S,13R,14Z,17S,18S,19R)- 2,13,18-tris(2-amino-2-oxoethyl)-7,12,17-tris(3- amino-3-oxopropyl)-3,5,8,8,13,15,18,19- octamethy1-2,7,12,17-tetrahydro-1H-corrin-21- id-3-yl]propanoylamino]propan-2-yl]phosphate; hydroxide | 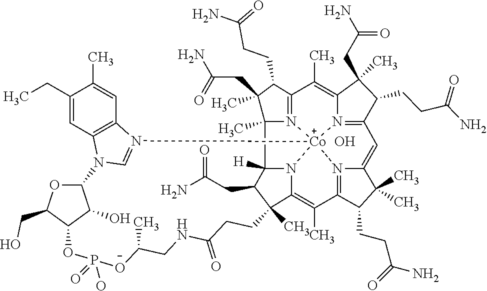 |

In one embodiment, the ready-to-use composition of the present invention has no more than 6.0% of total impurities in the composition at the end of 12 months, when stored at about 25° C. and 40% relative humidity.

In another embodiment, the ready-to-use composition of the present invention has no more than 0.5% of related B6-Hydroxymethyl derivative in the composition at the end of 12 months, when stored at about 25° C. and 40% relative humidity.

In yet another embodiment, the ready-to-use composition of the present invention has no more than 0.4% of related B5-Hydroxymethyl derivative in the composition at the end of 12 months, when stored at about 25° C. and 40% relative humidity.

In another embodiment, the ready-to-use composition of the present invention has no more than 1.0% of related C8-epimer of hydroxocobalamin in the composition at the end of 12 months, when stored at about 25° C. and 40% relative humidity.

In one embodiment, the ready-to-use composition of the present invention has no more than 1.0% of cyanocobalamin in the composition at the end of 12 months, when stored at about 25° C. and 40% relative humidity.

In another embodiment, the ready-to-use composition of the present invention has no more than 0.2% of unspecified impurity in the composition at the end of 12 months, when stored at about 25° C. and 40% relative humidity.

In one embodiment, the ready-to-use composition of the present invention has no more than 6.0% of total impurities in the composition at the end of 6 months, when stored at about 40±2° C. and not more than 25% relative humidity.

In another embodiment, the ready-to-use composition of the present invention has no more than 0.5% of related B6-Hydroxymethyl derivative in the composition at the end of 6 months, when stored at about 40-2° C. and not more than 25% RH.

In yet another embodiment, the ready-to-use composition of the present invention has no more than 0.4% of related B5-Hydroxymethyl derivative in the composition at the end of 6 months, when stored at about 40±2° C. and not more than 25% relative humidity.

In another embodiment, the ready-to-use composition of the present invention has no more than 1.0% of related C8-epimer of hydroxocobalamin in the composition at the end of 6 months, when stored at about 40±2° C. and not more than 25% relative humidity.

In one embodiment, the ready-to-use composition of the present invention has no more than 1.0% of cyanocobalamin in the composition at the end of 6 months, when stored at about 40±2° C. and not more than 25% relative humidity.

In another embodiment, the ready-to-use composition of the present invention has no more than 0.2% of unspecified impurity in the composition at the end of 6 months, when stored at about 40±2° C. and not more than 25% relative humidity.

Parenteral formulations tend to degrade over their shelf life due to permeation of oxygen into the parenteral solution. In the preferred embodiment of the present invention, the stable sterile aqueous ready-to-use composition of hydroxocobalamin contains not more than 5 ppm dissolved oxygen (DO), more preferably not more than 4 ppm dissolved oxygen (DO), more preferably not more than 3 ppm dissolved oxygen (DO), even more preferably not more than 2 ppm dissolved oxygen (DO), yet more preferably not more than 1.5 ppm dissolved oxygen (DO), and highly preferably not more than 1 ppm dissolved oxygen (DO).

In order to maintain low DO in the stable sterile aqueous ready-to-use composition of the present invention, the composition is packed in a primary packaging that is an oxygen impermeable flexible container, followed by packing it in a secondary packaging or pouch, in the presence of an oxygen absorber, which removes any oxygen that may be present. Alternatively, the primary packaging may be flushed with nitrogen, followed by packing in the secondary packaging with or without oxygen absorber.

Thus, the present invention provides containers/packaging for the hydroxocobalamin composition for parenteral use that is user-friendly, safe, flexible, easy to transport and store, compatible with the contents, minimizes extractables/leachables, is solvent-resistant, and durable. The ready-to-use composition is stored in a container comprising a primary packaging of polypropylene homopolymer. The compositions are stable and can be stored at 2-8° C. or at controlled room temperature.

In one embodiment, the stable sterile aqueous ready-to-use composition of the present invention is packaged in a container that comprises polypropylene bag with a twist off port as a primary packaging material. Alternatively, the primary packaging may be a polypropylene vial.

The ready-to-use composition of the present invention containing hydroxocobalamin or its pharmaceutically acceptable salt is stored in a primary packaging container made of polypropylene, surrounded by a secondary packaging which is a tri-laminated aluminium pouch of a size suitable for accommodating the primary packaging.

The present invention is further illustrated by reference to the following examples which are for illustrative purpose only and do not limit the scope of the invention in any manner.

### EXAMPLES

### Example 1

TABLE 3

| Ingredients | Quantity/mL | % w/v |
|---|---|---|
| Hydroxocobalamin | 25 mg | 2.5 |
| Sodium Chloride | 7 mg | 0.7 |
| Acetic acid (pH adjusting agent) | Q.S. to pH | Q.S. to pH |
| | 3.5 to 6.0 | 3.5 to 6.0 |
| Water for Injection | Q.S. to 1 mL | Q.S. to 100% |

All the ingredients as mentioned in Table 3 were dispensed as per the manufacturing formula. 120% of Water for Injection was taken in a manufacturing vessel as per batch quantity and purged with nitrogen to get the dissolved oxygen content to less than 2 ppm. 80% of Water for Injection required as per batch quantity was transferred into the compounding vessel. The required quantity of Sodium chloride was added into the above compounding vessel under stirring and mixed until it was completely dissolved. Hydroxocobalamin was then added into the above compounding vessel and mixed until it dissolved completely. The pH of the bulk solution thus obtained was measured and adjusted to pH 4.75 using 1M acetic acid solution, and it was mixed to get homogenous solution. The volume was made up to the batch size with water for injection previously purged with nitrogen, and stirred to get uniform homogenous solution.

The bulk solution was filtered through 0.22μ PVDF sterile filter and the solution was filled into 200 mL of Polypropylene Infusion bags. The bag was closed with a twist-off port. These filled bags were packed into aluminium pouches (tri-laminated) and the aluminium pouches were sealed properly.

### Example 2

The composition of Example 1 was charged for stability under two separate conditions of temperature and humidity and tested using the following method.

Stability testing-Assay-USP-NF
Chromatographic condition: A High-Performance Liquid Chromatograph (HPLC) Column: Supelco Chromolith® Performance RP-18e, 100×4.6 mm (Part No: 1021290001).
Two columns connected in series.
Test concentration: 110 μg/mL of hydroxocobalamin injection
Preparation of Mobile phase: 15.6 g/L of sodium dihydrogen phosphate is mixed in water and adjusted with phosphoric acid (1 in 100) to a pH of 3.0.
Diluent: Methanol, Buffer solution and water was prepared in the ratio 8:10:82.
Flow rate: 2.0 mL/min
Detection: UV 351 nm
Injection volume: 20 μL
Column oven temperature: 30° C.
Run time: 50 mins. (The retention time of hydroxocobalamin is about 17 min)
Acceptance criteria of the assay: 95.0%-115.0%

A gradient programme was run as per details in Table 4:

TABLE 4

| Time | Methanol | Buffer | Water |
|---|---|---|---|
| (min) | (%) | (%) | (%) |
| 0 | 8 | 10 | 82 |
| 20 | 8 | 10 | 82 |
| 40 | 40 | 10 | 50 |
| 45 | 8 | 10 | 82 |
| 50 | 8 | 10 | 82 |

Stability Testing-Related substances-USP-NF
Chromatographic condition: A High-Performance Liquid Chromatograph (HPLC)
Column: Supelco Chromolith® Performance RP-18e 100×4.6 mm (Part No: 1021290001).
Two columns connected in series.
RS concentration: 750 ppm
(Hydroxocobalamin Injection is a sterile solution of Hydroxocobalamin in Water for Injection.

It contains not less than 95.0 percent and not more than 115.0 percent of the labeled amount of hydroxocobalamin.) For system suitability 3.0 mL of Injection was diluted with pH 4.0 buffer (prepared by dissolving 2.61 g of sodium acetate and 20.5 g of sodium chloride in 5.25 mL of glacial acetic acid and sufficient water to make 1500 mL of solution) to 100 mL: the UV-visible absorption spectrum of this solution exhibits maxima at 352±2 nm and 525±2 nm. The ratio A/A is between 2.7 and 3.3.

Preparation of Mobile phase: 15.6 g/L of sodium dihydrogen phosphate is mixed in water and adjusted with phosphoric acid (1 in 100) to a pH of 3.0.
Diluent: Methanol, Buffer solution and water was prepared in the ratio 8:10:82.
Flow rate: 2.0 mL/min
Detection: UV 351 nm
Injection volume: 20 μL
Column oven temperature: 30° C.
Run time: 50 mins. (The retention time of hydroxocobalamin is about 17 min)
Acceptance criteria of the assay: not more than 4.0%.
Limit of quantification: 0.05%
Limit of detection: 0.0167%

A gradient programme was run as per details in Table 5:

TABLE 5

| Time | Methanol | Buffer | Water |
|---|---|---|---|
| (min) | (%) | (%) | (%) |
| 0 | 8 | 10 | 82 |
| 20 | 8 | 10 | 82 |
| 40 | 40 | 10 | 50 |
| 45 | 8 | 10 | 82 |
| 50 | 8 | 10 | 82 |

The stability results are tabulated in Table 6 below.

TABLE 6

| Condition | | 25 ± 2° C./40 ± 5% RH | | | | | 40 ± 2° C./NMT 25% RH | | | |
|---|---|---|---|---|---|---|---|---|---|---|
| Parameter | Specification | Initial | 1 M | 3 M | 6 M | 9 M | 1 M | 2 M | 3 M | 6 M |
| pH | 3.5 to 5.0 | 4.76 | 4.76 | 4.78 | 4.83 | 4.85 | 4.78 | 4.90 | 4.79 | 4.87 |
| Osmolality | 240 to 360 | 280 | 280 | 282 | 276 | 290 | 280 | 303 | 294 | 323 |
| (mOsm/kg) | | | | | | | | | | |
| Assay (%) | 95.0% to | 106.1 | 101.3 | 104.5 | 101.6 | 105.0 | 101.7 | 106.6 | 104.8 | 110.7 |
| | 115.0% | | | | | | | | | |

Related substances

| | | | | | | | | | | |
|---|---|---|---|---|---|---|---|---|---|---|
| B6- | NMT 0.50 | BQL | 0.05 | 0.05 | BQL | 0.05 | 0.05 | 0.05 | 0.05 | BQL |
| Hydroxymethyl | | | | | | | | | | |
| derivative | | | | | | | | | | |
| B5- | NMT 0.40 | BQL | BQL | BQL | BQL | BQL | BQL | BQL | BQL | BQL |
| Hydroxymethyl | | | | | | | | | | |
| derivative | | | | | | | | | | |
| Impurity At | NMT 0.30 | BQL | BQL | 0.07 | 0.08 | ND | BQL | BQL | 0.05 | 0.05 |
| RRT~0.70 | | | | | | | | | | |
| Impurity At | NMT 0.40 | BQL | ND | ND | BQL | 0.06 | ND | ND | ND | ND |
| RRT~0.75 | | | | | | | | | | |
| Impurity At | NMT 0.40 | BQL | BQL | BQL | 0.09 | 0.10 | BQL | BQL | BQL | 0.09 |
| RRT~0.82 | | | | | | | | | | |
| Impurity At | NMT 1.1 | BQL | BQL | 0.09 | 0.12 | 0.13 | 0.08 | 0.16 | 0.24 | 0.31 |
| RRT~0.93 | | | | | | | | | | |
| C8 Epimer | NMT 1.0 | ND | 0.05 | 0.12 | 0.28 | 0.42 | 0.12 | 0.39 | 0.57 | 1.17 |
| Impurity At | NMT 0.50 | ND | 0.40 | 0.49 | 0.28 | 0.60 | 0.88 | 0.89 | 1.60 | 1.28 |
| RRT~1.37-1.51 | | | | | | | | | | |
| Cyanocobalamin | NMT 1.0 | 0.90 | 0.82 | 0.89 | 0.73 | 0.91 | 0.84 | 0.71 | 0.93 | 0.82 |
| Unspecified | NMT 0.2 | 0.26 | 0.25 | 0.26 | 0.24 | 0.37 | 0.25 | 0.45 | 0.72 | 1.72 |
| Impurity | | | | | | | | | | |
| Total Impurities | NMT 4.0 | 1.88 | 1.79 | 2.38 | 2.61 | 3.55 | 2.83 | 4.25 | 6.11 | 9.24 |

RRT = Relative Retention Time;
NMT = Not More Than;
BQL-Below Quantification Level;
ND = Not Detected

The composition was found to have has not more than 4.0% of total impurities when stored for 3-9 months at about 25° C. and 40% RH, as well as when stored for 1-2 months at about 40° C. and 25% RH. Further, other impurities are also within the acceptable range as provided in Table 1 above.

### Example 3

Batch 2 of WO2022063963 was prepared as described in the publication. The stability data of this example of WO2022063963 is compared with stability data of the composition of Example 1 of the present invention. The comparison of both the compositions with their stability data are provided in Tables 7 and 8 below, respectively. It must be noted that example of WO2022063963 only provides a hydroxocobalamin composition of strength 0.02% w/v, but the comparison is done as this was the closest prior art. Note also that nothing in WO2022063963 teaches or suggests solution compositions of hydroxocobalamin with higher strength, nor any compositions without the use of solubilizing agents such as PVP K30 and EDTA. The compositions of the present invention are different in that no solubilizing agent or chelating agent is used.

TABLE 7

| | Batch 2 of | |
|---|---|---|
| | WO2022063963 | Example 1 |
| Ingredients | % w/v | % w/v |
| Hydroxocobalamin | 0.02 | 2.5 |
| Sodium chloride | — | 0.7 |
| PVP K30 | 0.05 | — |
| EDTA | 0.01 | — |
| Water for Injection | Qs to 100% | Qs to 100% |
| Acetic acid | — | Qs to pH 4.75 |
| Hydrochloric acid/Sodium | Qs to pH 5.9 | — |
| hydroxide | | |

TABLE 8

| | Batch 2 of WO2022063963 | | | Example 1 | | |
|---|---|---|---|---|---|---|
| | Condition: | | | Condition: | | |
| | 40° C. ± 2° C. | | | 40° C. ± 2° C. | | |
| Test Parameter | Initial | 1 week | 2 week | Initial | 1 week | 2 week |
| pH | 5.99 | 6.24 | 6.12 | 4.82 | 4.75 | 4.78 |
| Osmolality | 11 | 10 | 13 | 300 | 294 | 305 |
| Assay | 96.3 | 90.5 | 88.8 | 102 | 99.4 | 102.7 |

Related substances

| | | | | | | |
|---|---|---|---|---|---|---|
| B6- | BQL | BQL | 0.05 | BQL | 0.07 | 0.08 |
| Hydroxymethyl | | | | | | |
| derivative | | | | | | |
| B5- | BQL | BQL | 0.06 | BQL | BQL | BQL |
| Hydroxymethyl | | | | | | |
| derivative | | | | | | |
| Impurity @ | BQL | ND | ND | ND | ND | ND |
| RRT~0.70 | | | | | | |
| Impurity @ | BQL | ND | ND | BQL | ND | ND |
| RRT~0.75 | | | | | | |
| Impurity @ | BQL | ND | ND | ND | 0.05 | BQL |
| RRT~0.82 | | | | | | |
| Impurity @ | 0.12 | 0.22 | 0.23 | ND | ND | 0.11 |
| RRT~0.93 | | | | | | |
| C8 Epimer | 0.13 | 0.53 | 1.37 | 0.14 | 0.14 | 0.17 |
| Impurity @ RRT | 0.30 | 0.51 | 0.47 | 0.54 | 0.67 | 0.70 |
| 1.37-1.51 | | | | | | |
| Cyanocobalamin | 0.41 | 0.47 | 0.55 | 0.50 | 0.47 | 0.46 |
| Total Impurities | 1.43 | 2.21 | 3.19 | 1.67 | 1.87 | 2.20 |

RRT = Relative Retention Time;
NMT = Not More Than;
BQL-Below Quantification Level;
ND = Not Detected

As can be seen from the stability results in Table 8 above, the total impurities for Batch 2 of WO2022063963 are higher than that for Example 1 of the present invention at the end of 2 weeks. Similarly, the C8-epimer impurity is higher for Batch 2 of WO2022063963. It can therefore be concluded that the composition of the present invention is more stable than the prior art example of WO2022063963, despite having a higher concentration (strength) and despite having no solubilizers therein.

### Example 4

Provided herein are the stability test results of 4 batches of stable sterile aqueous ready-to-use compositions of hydroxocobalamin in Polypropylene bags and vials, across different conditions of temperature and relative humidity. All 4 batches were prepared as per Example 1 above. The batch 1 was stored in 200 ml polypropylene bag with a fill volume of 200 ml, at controlled room temperature of about 25° C. and about 40% RH. The results of stability testing are shown in Table 10 below

TABLE 10

| Condition | | 25° C./40% RH | | | | | |
|---|---|---|---|---|---|---|---|
| Parameter | Specification | Initial | 1 M | 3 M | 6 M | 9 M | 12 M |
| pH | 3.5-6.0 | 4.72 | 4.75 | 4.85 | 4.77 | 4.75 | 4.74 |
| Osmolality | 250-330 | 294 | 307 | 299 | 298 | 303 | 301 |
| (mOsmol/kg) | | | | | | | |
| Assay (%) | 95-115 | 101.4 | 102.8 | 99.9 | 97.1 | 97.5 | 96.9 |
| Weight loss (%) | NMT 5% | NA | −0.04 | 0.02 | −0.012 | -0.013 | −0.007 |

Related substances (% w/w)

| | | | | | | | |
|---|---|---|---|---|---|---|---|
| B6-Hydroxymethyl | NMT 0.50% | 0.04 | 0.03 | 0.07 | 0.04 | 0.03 | 0.07 |
| derivative | | | | | | | |
| B5-Hydroxymethyl | NMT 0.50% | 0.03 | 0.02 | ND | 0.04 | 0.03 | 0.04 |
| derivative | | | | | | | |
| Impurity @ | NMT 0.30 | ND | ND | ND | ND | 0.05 | ND |
| RRT~0.70 | | | | | | | |
| Impurity @ | NMT 0.40 | ND | ND | ND | ND | ND | ND |
| RRT~0.75 | | | | | | | |
| Impurity @ | NMT 0.40% | 0.07 | 0.08 | ND | 0.06 | 0.06 | ND |
| RRT~0.84 | | | | | | | |
| Impurity @ | NMT 1.1% | 0.07 | 0.05 | ND | 0.08 | 0.13 | 0.13 |
| RRT~0.93 | | | | | | | |
| C8 Epimer | NMT 1.0% | 0.06 | 0.03 | 0.10 | 0.27 | 0.29 | 0.33 |
| Impurity @ RRT | NMT 0.50% | 0.15 | 0.22 | 0.25 | 0.33 | 0.30 | 0.42 |
| 1.37-1.51 | | | | | | | |
| Cyanocobalamin | NMT 1.0% | 0.45 | 0.40 | 0.48 | 0.46 | 0.43 | 0.50 |
| Any Unspecified | NMT 0.2% | 0.09 @ | 0.07 @ | 0.07 @ | 0.12 @ | 0.11 @ | 0.11 @ |
| Impurity | | RRT | RRT | RRT | RRT | RRT | RRT |
| | | 0.48 | 0.48 | 0.48 | 1.87 | 1.73 | 1.62 |
| Total Impurities | NMT 6.0% | 1.6 | 1.44 | 1.94 | 2.66 | 3.25 | 4.07 |

RRT = Relative Retention Time;
NMT = Not More Than;
BQL-Below Quantification Level;
ND = Not Detected

The batch 2 was stored in 200 ml polypropylene bag with a fill volume of 200 ml, at controlled room temperature of about 25° C. and about 40% RH. The results of stability testing are shown in Table 11 below.

TABLE 11

| Condition | | 25° C./40% RH | | | |
|---|---|---|---|---|---|
| Parameter | Specification | Initial | 1M | 2M | 3M |
| pH | 3.5-6.0 | 4.77 | 4.79 | 4.82 | 4.81 |
| Osmolality (mOsmol/kg) | 250-330 | 286 | 319 | 317 | 309 |
| Assay (%) | 95-115 | 102.9 | 103.7 | 100.4 | 99.9 |
| Weight loss (%) | NMT 5% | NA | 0.019 | 0.016 | 0.035 |

Related substances (% w/w)

| | | | | | |
|---|---|---|---|---|---|
| B6-Hydroxymethyl | NMT 0.50% | 0.02 | 0.11 | 0.02 | 0.06 |
| derivative | | | | | |
| B5-Hydroxymethyl | NMT 0.50% | 0.02 | 0.04 | 0.02 | 0.10 |
| derivative | | | | | |
| Impurity @ RRT ~0.70 | NMT 0.30 | ND | 0.05 | ND | ND |
| Impurity @ RRT ~0.75 | NMT 0.40 | 0.03 | 0.05 | ND | ND |
| Impurity @ RRT~0.82 | NMT 0.40% | 0.05 | ND | ND | ND |
| Impurity @ RRT~0.93 | NMT 1.1% | 0.14 | 0.15 | 0.15 | 0.15 |
| C8 Epimer | NMT 1.0% | 0.07 | 0.16 | 0.15 | 0.16 |
| Impurity @ RRT 1.37-1.51 | NMT 0.50% | 0.23 | 0.27 | 0.26 | 0.34 |
| Cyanocobalamin | NMT 1.0% | 0.84 | 0.87 | 0.79 | 0.83 |
| Any Unspecified Impurity | NMT 0.2% | 0.09 | 0.10 | 0.10 | 0.10 |
| | | @RRT0.48 | @RRT1.52 | @RRT1.69 | @RRT1.89 |
| Total Impurities | NMT 7.0% | 2.02 | 2.46 | 2.57 | 2.66 |

RRT = Relative Retention Time;
NMT = Not More Than;
BQL—Below Quantification Level;
ND = Not Detected

The batch 3 was stored in 200 ml polypropylene bag with a fill volume of 200 ml, at controlled room temperature of about 25° C. and about 40% RH and at about 2-8° C. for a period of one month. The results of stability testing of batch 3 are shown in Table 12 below:

TABLE 12

| | | | | 25 ± 2° C./ |
|---|---|---|---|---|
| Condition | | | 2-8° C. | 40 ± 5% RH |
| Parameter | Specification | Initial | 1M | 1M |
| pH | 3.5-6.0 | 4.97 | 4.93 | 4.93 |
| Osmolality | 250-330 | 280 | 285 | 256 |
| (mOsmol/kg) | | | | |
| Assay (%) | 95-115 | 103.2 | 101.3 | 102.3 |
| Weight loss (%) | NMT 5% | NA | 0.000 | −0.009 |

Related substances (% w/w)

| | | | | |
|---|---|---|---|---|
| B6-Hydroxymethyl | NMT 0.50% | 0.03 | 0.07 | 0.07 |
| derivative | | | | |
| B5-Hydroxymethyl | NMT 0.50% | ND | 0.05 | 0.06 |
| derivative | | | | |
| Impurity @ RRT ~0.70 | NMT 0.30 | ND | ND | ND |
| Impurity @ RRT ~0.75 | NMT 0.40 | 0.05 | ND | ND |
| Impurity @ RRT~0.82 | NMT 0.40% | ND | ND | ND |
| Impurity @ RRT~0.93 | NMT 1.1% | 0.25 | 0.26 | 0.27 |
| C8 Epimer | NMT 1.0% | 0.15 | 0.17 | 0.19 |
| Impurity @ RRT 1.37- | NMT 0.50% | 0.37 | 0.51 | 0.46 |
| 1.51 | | | | |
| Cyanocobalamin | NMT 1.0% | 0.63 | 0.67 | 0.67 |
| Any Unspecified | NMT 0.2% | 0.15 | 0.09 | 0.11 |
| Impurity | | | | |
| Total Impurities | NMT 7.0% | 1.98 | 2.38 | 2.44 |

RRT = Relative Retention Time;
NMT = Not More Than;
BQL—Below Quantification Level;
ND = Not Detected

The batch 4 was stored in 200 ml polypropylene vial with a fill volume of 200 ml, at controlled room temperature of about 25° C. and about 40% RH and at about 2-8° C. for a period of one month. The results of stability testing of the batch are shown in table 13 below:

TABLE 13

| | | | | 25 ± 2° C./ |
|---|---|---|---|---|
| Condition | | | 2-8° C. | 40 ± 5% RH |
| Parameter | Specification | Initial | 1M | 1M |
| pH | 3.5-6.0 | 4.91 | 4.87 | 4.9 |
| Osmolality | 250-330 | 287 | 292 | 295 |
| (mOsmol/kg) | | | | |
| Assay (%) | 95-115 | 103.5 | 103.1 | 103.3 |
| Weight loss (%) | NMT 5% | NA | −0.008 | 0.023 |

Related substances (% w/w)

| | | | | |
|---|---|---|---|---|
| B6-Hydroxymethyl | NMT 0.50% | 0.02 | 0.07 | 0.06 |
| derivative | | | | |
| B5-Hydroxymethyl | NMT 0.50% | 0.03 | 0.05 | 0.04 |
| derivative | | | | |
| Impurity @ RRT ~0.70 | NMT 0.30 | ND | ND | ND |
| Impurity @ RRT ~0.75 | NMT 0.40 | ND | ND | ND |
| Impurity @ RRT~0.82 | NMT 0.40% | ND | ND | ND |
| Impurity @ RRT~0.93 | NMT 1.1% | 0.23 | ND | 0.2 |
| C8 Epimer | NMT 1.0% | 0.18 | 0.22 | 0.16 |
| Impurity @ RRT 1.37- | NMT 0.50% | 0.26 | 0.41 | 0.46 |
| 1.51 | | | | |
| Cyanocobalamin | NMT 1.0% | 0.75 | 0.91 | 0.77 |
| Any Unspecified | NMT 0.2% | 0.15 | 0.15 | 0.15 |
| Impurity | | | | |
| Total Impurities | NMT 7.0% | 1.97 | 2.56 | 2.40 |

RRT = Relative Retention Time;
NMT = Not More Than;
BQL—Below Quantification Level;
ND = Not Detected

We claim:

1. A stable sterile aqueous ready-to-use pharmaceutical composition for parenteral use comprising:
a) hydroxocobalamin or its pharmaceutically acceptable salt as the sole active ingredient;
(b) a tonicity adjusting agent in an amount sufficient to provide osmolality of about 240 mOsm/kg to about 360 mOsm/kg; and
(c) a pH modifying agent;
wherein upon storage of the composition in a container comprising a primary packaging of polypropylene homopolymer at controlled room temperature or at 2-8° C. for 18 months, the composition has—
(i) not more than about 0.5% of impurity B6-Hydroxymethyl derivative,
(ii) not more than about 0.4% of impurity B5-Hydroxymethyl derivative,
(iii) not more than about 1% of C8-epimer impurity,
(iv) not more than about 1% of cyanocobalamin,
(v) not more than about 0.2% of any unspecified impurity, and
(vi) not more than about 6% of total impurities.

2. The pharmaceutical composition as claimed in claim 1, wherein hydroxocobalamin or its pharmaceutically acceptable salt is present in the range of about 0.1% w/v to about 5% w/v of the composition.

3. The pharmaceutical composition as claimed in claim 1, wherein the tonicity adjusting agent is selected from the group comprising sodium chloride, potassium chloride, mannitol, lactose, dextrose, glycerol, and mixtures thereof.

4. The pharmaceutical composition as claimed in claim 3, wherein the tonicity adjusting agent is sodium chloride.

5. The pharmaceutical composition as claimed in claim 3, wherein the tonicity adjusting agent is present in an amount ranging from about 0.5% w/v to about 1% w/v of the composition.

6. The pharmaceutical composition as claimed in claim 1, wherein the pH modifying agent is selected from the group comprising acetic acid, tartaric acid, lactic acid, hydrochloric acid, citric acid, succinic acid, fumaric acid, maleic acid, malic acid, gluconic acid, their pharmaceutically acceptable salts and mixtures thereof.

7. The pharmaceutical composition as claimed in claim 6, wherein the pH modifying agent is acetic acid.

8. The pharmaceutical composition as claimed in claim 6, wherein the pH of the composition is in the range of about 3.5 to about 6.

9. The pharmaceutical composition as claimed in claim 1, wherein the composition is essentially free of preservatives, co-solvents, polymers, chelating agents and reconstitution or dilution agents.

10. The pharmaceutical composition as claimed in claim 1, wherein the composition contains not more than 5 ppm of dissolved oxygen.

11. The pharmaceutical composition as claimed in claim 1, wherein the primary packaging is a polypropylene bag with a twist off port or a polypropylene vial.

12. The pharmaceutical composition as claimed in claim 1, wherein the primary packaging is surrounded by a secondary packaging material consisting of tri-laminated aluminum pouch.

13. The pharmaceutical composition as claimed in claim 1, wherein the composition contains 25 mg/ml of hydroxocobalamin or its pharmaceutically acceptable salt.
